(19) United States
(12) Patent Application Publication
(10) Pub. No.: US 2014/0316455 A1
(43) Pub. Date: Oct. 23, 2014

(54) SYSTEM AND METHOD FOR PERFORMING SURGICAL PROCEDURES

(71) Applicants: Swaminadhan GNANASHANMUGAM, San Francisco, CA (US); Peter Franklin CAMPBELL, Newark, CA (US); Neel B. PATEL, Chicago, IL (US)

(72) Inventors: Swaminadhan GNANASHANMUGAM, San Francisco, CA (US); Peter Franklin CAMPBELL, Newark, CA (US); Neel B. PATEL, Chicago, IL (US)

(21) Appl. No.: 14/213,103

(22) Filed: Mar. 14, 2014

Related U.S. Application Data
(60) Provisional application No. 61/800,144, filed on Mar. 15, 2013.

(51) Int. Cl. A61B 19/00; A61B 17/135; A61B 19/04; A61B 19/02

(52) U.S. Cl. CPC A61B 19/38; A61B 19/026; A61B 17/135; A61B 19/04; A61B 2019/385; A61B 2019/0267; A61B 2019/4868; A61B 2017/0023
USPC 606/202; 606/1; 128/897

(57) ABSTRACT

A method for performing a surgical or medical procedure on a portion of a patient's body within a surgical environment enclosure may involve preparing the surgical environment enclosure for performing the procedure, advancing the portion of the patient's body into the surgical environment enclosure through a first port on the enclosure, and performing the surgical or medical procedure on the portion of the patient's body inside the surgical environment enclosure, through at least a second port on the enclosure. The first port forms a seal around a surface of the patient's body upon or after insertion. In some embodiments, neither the entire body of the patient nor an entire body of any medical or surgical personnel fully enters the surgical environment enclosure during performance of the surgical or medical procedure.

### CROSS-REFERENCE TO RELATED APPLICATION(S)

This application claims priority to U.S. Provisional Patent Application Ser. No. 61/800,144, entitled “System and Method for In-Office Surgery,” filed Mar. 15, 2013. The full disclosure of the above-listed patent application is hereby incorporated by reference herein.

### FIELD

The present application relates to medical and surgical methods and systems. More specifically, the present application relates to a method and system for providing an environment in which to perform a surgical or medical procedure.

### BACKGROUND

Surgical procedures take various different forms and entail differing levels of resource requirements. In the US, approximately 30-40 million surgeries are performed annually, many of them requiring the use of an operating room and associated staff, facilities, and resources. Operating rooms are typically located in a hospital or an outpatient surgical center, which means that surgical procedures performed in operating rooms also incur additional resource use involved in using these settings for healthcare delivery. As such, surgery tends to be quite resource intensive and expensive to perform, an increasingly important problem given escalating health care costs.

Even surgical procedures performed in traditional operating rooms have variable rates of surgical site infection, depending on the area of the body operated upon. Increasingly complex hospital systems with increasing operating room personnel traffic (turnover of nurse and other staff, for example) have actually resulted in increasing infection rates. A sterile environment conducive to performing a surgical procedure is produced through a variety of steps, procedures and protocols that involve the sterile method or fashion, also referred to as aseptic surgical techniques or procedures.

In general, the principles of aseptic technique for surgery involve ensuring that only sterile objects are involved in the procedure. That sterility is maintained by ensuring that only sterile or sterilized objects touch sterile objects, ensuring that the patient's operative site is appropriately cleaned and the microbial load eliminated or reduced as much as possible with the appropriate cleaning agents and sterile draping pre-procedure, ensuring that all instruments, surgeons, and staff that come into contact with the operative site are also sterile, and further procedures that are involved in maintaining a sterile operating room, for example the use of laminar air flow within the operating room to minimize any further microbial contamination of the operative field. In essence, the principles involve disinfecting the patient, disinfecting the staff, disinfecting any and all instruments that come into contact with the patient and the staff, and taking control procedures to ensure that the operating room and field remain sterile for the duration of the surgical procedure.

Despite best efforts, surgical site infections still occur, with varying rates of infection depending on the type of surgery and the patient that is involved. An increase in the number of operating room personnel is one contributing factor, as five or more personnel are known to increase the microbial load by 15-fold or more. Throughout an operation, the presence of five different personnel is increasingly more common, given staff turnover and increased traffic by persons in and out of the operating room.

Clean air technologies can help reduce surgical site infections and maintain aseptic technique throughout the procedure. Horizontal airflow units, for example, are known to reduce airborne contamination of the surgical site. However, with increasing OR traffic and personnel exchanges, the actual maintenance of laminar airflow is often suboptimal, as it is decreased and adversely affected by the OR traffic. Furthermore, surgical instruments, when opened in a clean air environment, have a 28-fold decreased bacterial fallout when compared to a conventional MOM.

Ultraviolet light is another technology that can be used to reduce bacterial contamination in a surgical wound. However, ultraviolet light is not typically available or used in current practice. Surgical isolator gowns, which offer increased protection of staff and reduce bacterial counts as afforded by conventional procedures such as surgical scrubbing, masks, gowns, and gloves, are also not used frequently or ubiquitously in operating rooms, due to costs and inconvenience among other concerns. Finally, though a patient is well prepped and draped, oftentimes initially at the time of the surgery, the continued assailment of the surgical site by the aforementioned avenues for contamination continues to pose a problem, despite best efforts to maintain a clean, aseptic, low microbial load surgical site throughout the duration of the operation.

Furthermore, the most commonly used and available setting that currently exists for performing relatively aseptic surgical procedures is the operating room. As mentioned above, operating rooms, while not completely effective in preventing all surgical site infections, still remain the gold standard venue for performing surgical procedures. However, operating rooms are very expensive to maintain, run, and use. For example, a typical operating room may cost as much as $30-$60 per minute to use, or even higher. Additionally, operating rooms often involve the use of many staff members, including a circulating nurse, scrub nurse or scrub tech, surgeon, operative assistant, anesthesia personnel, with personnel costs increasing the costs of the overall operation. While there is some variation in operating rooms and their design, many operating rooms are general-purpose operating rooms, catering to the needs of many different types of operations. As such, their costs tend to be higher, as they need to fulfill differing requirements. Furthermore, operating rooms tend to be located in hospitals or in outpatient surgical centers. Thus, performing an operation incurs associated costs involved with the running and maintenance of that healthcare provider environment, i.e. the hospital or the surgical center. The need to “turn over” an operating room, i.e. to complete an operation, move the patient out of the operating room, clean the operating room, set up for the next operation, and then bring in the next patient and begin the process of setting up for the next operation, also leads to increased costs, as the process requires significant “dead time.”

With escalating healthcare costs in the U.S., accounting for 15% of GDP or higher, any methods or systems for decreasing healthcare costs while maintaining or increasing quality of care are very desirable. In international settings outside the U.S., cost concerns and the need for savings may be even more pressing.

It would be very advantageous, therefore, to have methods and systems for performing certain surgical interventions in less expensive settings than a full operating room facility. Ideally, such methods and systems would reduce resource use and costs, while still maintaining the essential functionality of an operating room, such as providing a controlled, sterile environment.

It would also be highly advantageous to have methods and systems for reducing surgical infections generally, whether the surgical procedures are performed in the operating room or elsewhere. Ideally, such methods and systems would reduce infection rates as compared to traditional operating rooms. Alternatively, such methods and systems would still be advantageous if they resulted in infection rates comparable to those of traditional operating rooms but at a lower cost.

### BRIEF SUMMARY

The systems, devices and methods described herein generally provide a surgical environment outside of an operating room. For the purposes of this document, surgical environment broadly encompasses any environment in which surgery and/or any other therapeutic procedure or intervention and/or diagnostic procedure or intervention is performed, where sterile technique would be preferable or advantageous. For example, some embodiments of a surgical environment enclosure system described herein may be used in a physician's clinic or similar setting. In many embodiments, the systems, devices and methods are described for use in performing surgical procedures on the extremities (hand, arm, foot, leg, etc.). For illustrative purposes, the method and associated devices are specifically described in the setting of hand surgery, specifically for carpal tunnel surgery. However, in the embodiments described or in alternative embodiments, the described systems, devices and methods may be used for any suitable interventional or surgical procedure on any extremity or even on other areas of the body.

In one embodiment, a method involves prepping and draping a patient's forearm and hand according to the traditional surgical method, and then inserting the patient's hand/forearm into a surgical environment device that forms an aseptic, sterile, surgical environment around the hand. Once the hand is located within the device, a tourniquet or other sealing mechanism is deployed to create a seal or a substantial seal around the patient's extremity that will prevent or reduce any outside bacterial contamination into the aseptic, sterile surgical environment. In some embodiments, prepackaged surgical instruments will be provided within the surgical field, to facilitate performing the surgical procedure, such as a scalpel and other instruments for performing carpal tunnel surgery, in this example. In other embodiments, these instruments will similarly be introduced into the surgical environment as and when desired, for example, via a temporary opening in the surgical environment that can be opened and closed when necessary.

The surgical environment enclosure system will also include hand ports through which a surgeon (and in some cases, his or her assistant(s)) can advance his/her hands, forearm, and extremities to perform the surgical procedure. The surgical environment enclosure may be made of translucent plastic, glass or other clear material, to allow for visualization through the walls of the environment and allow for sufficient visualization to perform surgery. Alternative embodiments may include “seeing windows” for use within the environment or the use of endoscopic or laparoscopic type camera based visualization to facilitate the performance of surgery, or any combination thereof.

In some embodiments, the surgical environment enclosure may also include a high-efficiency particulate absorption (“HEPA”) filter or other air-filtration laminar flow system, to maintain clean air flow, horizontal laminar flow, positive pressure or other air flow pattern within the surgical environment, to facilitate lower bacterial loads upon the surgical site. In some embodiments, the air flow unit may be separate from the surgical environment and may plug into the surgical environment through an additional port. In addition, a means for interfacing with ultraviolet light may also be included, either as a built-in unit into the surgical environment, as a potential add-on attachment that is interfaced onto the surgical environment, as a plug-in from the external environment, or other method. The surgical environment may also contain interfaces for surgical instruments, such as cautery instrumentation, suction, irrigation and the like, to facilitate their use within the surgical environment without contaminating the environment.

In one aspect, a method for performing a surgical or medical procedure on a portion of a patient's body within a surgical environment enclosure may involve: preparing the surgical environment enclosure for performing the procedure; advancing the portion of the patient's body into the surgical environment enclosure through a first port on the enclosure, wherein the first port forms a seal around a surface of the patient's body; and performing the surgical or medical procedure on the portion of the patient's body inside the surgical environment enclosure, through at least a second port on the enclosure. In some embodiments, neither the entire body of the patient nor an entire body of any medical or surgical personnel fully enters the surgical environment enclosure during performance of the surgical or medical procedure.

In some embodiments, preparing the surgical environment enclosure may involve expanding the enclosure from a collapsed configuration to an expanded configuration. In some embodiments, preparing the surgical environment enclosure may involve removing the enclosure from sterile packaging before expanding the enclosure. In some embodiments, expanding the enclosure may involve releasing the enclosure from the sterile packaging and allowing the enclosure to automatically expand to a default configuration when released from constraint. In other embodiments, expanding the enclosure may involve inflating a portion of the enclosure.

Preparing the surgical environment enclosure may also involve establishing an air flow through the enclosure by passing air into the enclosure through a first ventilation port and passing air out of the enclosure through a second ventilation port. Preparing the surgical environment enclosure may also involve passing at least one sterile instrument into the enclosure. Alternatively, in some embodiments, preparing the surgical environment enclosure does not require passing any instruments into the enclosure, because the enclosure is provided with at least one instrument prepackaged within it.

In some embodiments, advancing the portion of the patient's body into the enclosure may involve passing an extremity of the patient's body at least partway into the enclosure. In various embodiments, the extremity may be an arm or a leg. In one embodiment, the arm may be passed into the enclosure, and the procedure may be a carpal tunnel release procedure.

Some embodiment may further involve forming a blood flow limiting compression around the surface of the patient's body using a compression member coupled with the first port. Performing the procedure typically involves extending two hands of a surgeon into the enclosure through the second port and a third port on the enclosure. Performing the procedure may further include extending two hands of an assistant into the enclosure through a fourth port and a fifth port on the enclosure. Passing the hands of the surgeon into the enclosure may involve passing the hands into gloves that are attached to the second and third ports. Performing the procedure may also involve passing a working distal end of at least one instrument through an instrument port on the enclosure, where a proximal end of the at least one instrument remains outside the enclosure for grasping and manipulating by a surgeon or assistant.

The method may also include, after performing the procedure, collapsing the enclosure and disposing of the enclosure. Collapsing the enclosure may involve, for example, removing air from a portion of the enclosure. Disposing of the enclosure may involve, for example, mailing the enclosure to a facility for disposal. The method may also optionally include, after performing the procedure, sterilizing the enclosure and reusing the enclosure for an additional procedure. Furthermore, preparing the surgical environment enclosure may include sterilizing an internal surface of the enclosure.

In another aspect, a surgical environment enclosure system for performing a surgical or medical procedure on a portion of a patient's body may include: an enclosure; a patient port on the enclosure for allowing passage of the portion of the patient's body into the enclosure; at least two hand ports on the enclosure for allowing passage of two physician hands into the enclosure to perform the procedure; a first ventilation port on the enclosure for allowing controlled passage of air into the enclosure; and at least a second ventilation port on the enclosure for allowing controlled passage of air out of the enclosure. The patient port includes a seal-forming member for forming a seal with a surface of the patient's body to prevent air from passing into the enclosure through the patient port while the portion of the patient's body is in the enclosure.

In some embodiments, the enclosure may be collapsible and expandable. In some embodiments, the enclosure, when in an expanded state, is at least partially rigid. The enclosure may be made of a transparent material. An internal surface and/or an external surface of the enclosure may be provided in a sterile or near-sterile condition. Some embodiments include four hand ports for allowing passage of two physician hands and two assistant hands into the enclosure. Other embodiments may include more than four hand ports. In some embodiments, at least some of the hand ports may be moveable along a surface of the enclosure to allow the physician's hands to move in a direction parallel to the surface of the enclosure. For example, such an embodiment may include a track in the surface of the enclosure, where the hand ports move along the track in at least two directions along the surface.

In some embodiments, the system may further include at least two built-in gloves, where each of the two hand ports is attached to one of the built-in gloves extending into the enclosure such that when one of the hands passes through one of the hand ports it passes immediately into the glove attached to that port. Some embodiments may further include a circumferential compression member coupled with the patient port to apply circumferential compression to a surface of the patient's body and thus limit blood flow to the portion of the patient's body located within the enclosure. Some embodiments may further include an air input tube removably coupled with the first ventilation port and an air evacuation tube removably coupled with the second ventilation port. Some embodiments may further include an air circulation device coupled with the air input tube and the air evacuation tube for providing air to, and removing air from, the enclosure. For example, such an air circulation device may include a high-efficiency particulate absorption filtration system.

In some embodiments, the system may also include at least one instrument port on the enclosure for allowing at least one surgical instrument to be passed therethrough, where the at least one instrument port is configured to form a seal with the at least one surgical instrument. In some embodiments, the system may include at least one instrument prepackaged within the enclosure for use in the medical or surgical procedure.

These and other aspects and embodiments will be described in further detail below, in reference to the attached drawing figures.

### BRIEF DESCRIPTION OF DRAWINGS

FIGS. 1A and 1B are side perspective and bottom perspective views, respectively, of a surgical environment enclosure system, according to one embodiment;

FIGS. 2A and 2B are perspective views of a surgical environment enclosure system, with FIG. 2B illustrating the system in use, according to an alternative embodiment;

FIG. 3 is a perspective view of a surgical environment enclosure system in use, according to another alternative embodiment;

FIG. 4 is a perspective view of a surgical environment enclosure system in use, according to another alternative embodiment;

FIG. 5 is a perspective view of a surgical environment enclosure system in use, according to another alternative embodiment;

FIG. 6 is a perspective view of a surgical environment enclosure system sized for entry by a patient and multiple physicians, according to another alternative embodiment;

FIG. 7 is a perspective view of a surgical environment enclosure system sized for entry by a patient and multiple physicians, according to another alternative embodiment; and

FIG. 8 is a perspective view of a helicopter transporting a packaged surgical environment enclosure system, illustrating one transport method, according to another alternative embodiment.

### DETAILED DESCRIPTION

The following description explains details of several different embodiments of a method and system for performing medical or surgical procedures in a surgical environment enclosure. The enclosure systems described herein are generally smaller and less expensive to build and use than currently available operating rooms. In some cases, in fact, the enclosure systems are significantly smaller than a conventional operating room and define a space in which a portion of a patient's body can be operated on by a physician in a confined environment under sterile or relatively clean, germ free conditions, at a fraction of the cost of building and/or maintaining an operating room. Such embodiments may be used to perform medical or surgical procedures in any of a number of locations, such as but not limited to a physician's office or treatment room, an emergency room, a treatment room in a hospital that is not certified as an operating room, a remote location, a field location such as a battlefield or disaster zone, or the like.

In addition to being useful in a number of possible locations, the systems and methods described herein may also be used for any of a number of different medical or surgical procedures. In some embodiments, for example, the system and method may be used for surgical procedures on the upper and/or lower limbs. In one specific example, the system and method are used for hand surgery procedures and specifically for carpal tunnel release surgery. In other embodiments, the system and method may be used for foot, ankle and/or leg surgery. In these limb surgery examples, the system may be quite small, and a portion of the limb may be inserted into the surgical environment enclosure for performance of the procedure. In other examples, it may be possible to position the surgical enclosure on a patient's torso, and a procedure may be performed through the enclosure. In the largest examples, the enclosure may be large enough for a whole patient, a physician, and at least one assistant to enter fully for performing the procedure. Generally, any surgical or medical procedure, whether therapeutic, diagnostic or both, may be performed in any setting, according to various alternative embodiments.

These and other features and examples will be described in further detail below. The embodiments discussed herein are provided for exemplary purposes only and should not be interpreted as limiting the scope of this application as set forth in the claims.

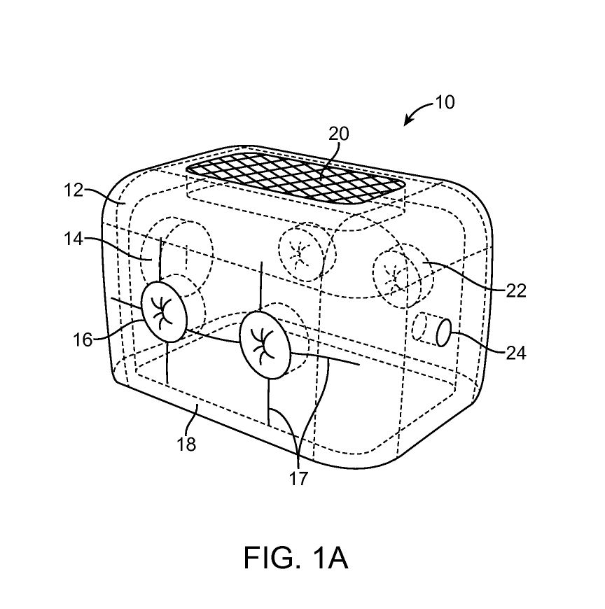

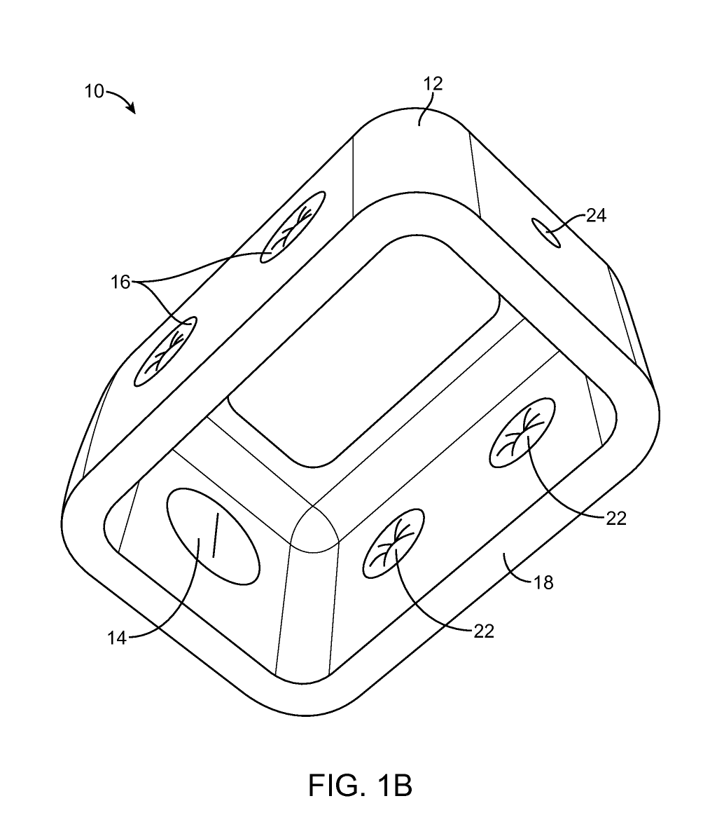

Referring now to FIGS. 1A and 1B, one embodiment of surgical environment enclosure system 10 may include a surgical environment enclosure 12, which in this embodiment is an approximately rectangular, box-like enclosure that is expandable and collapsible and has a base 18. Enclosure 12, in this embodiment, is configured for performing a medical or surgical procedure on a limb of a patient. In alternative embodiments, enclosure 12 may have a fixed shape, such as that shown in FIGS. 1A and 1B, without being collapsible or expandable. An expandable/collapsible embodiment, however, may be advantageous for storage and/or shipping purposes. Enclosure 12 will typically be made of a clear, polymeric material, so that it will be partially or completely transparent to facilitate visualization of the surgical field. Alternatively, in some embodiments, such as when collapsibility is not important and/or if enclosure 12 is a more permanent structure, enclosure 12 may be made entirely or partially of glass or some other non-flexible, transparent material. In other embodiments, only a portion of enclosure 12 may be transparent, for example including a window through which the procedure is viewed by the physician. Generally, however, maximizing visibility is preferred.

Base 18 generally refers to the lower surface of enclosure 12. In various alternative embodiments, base 18 may be made of various materials and/or have various thicknesses, sizes or configurations. In some embodiments, base 18 is a continuous surface and remains completely intact during a surgical procedure, thus maintaining enclosure 12 as a six-sided, box-like enclosure thorough the procedure. In alternative embodiments, base 18 may form only a partial bottom for enclosure 12, thus leaving a portion open, through which a portion of a patient's body (abdomen, thorax, back, etc.) may be accessed for performing a procedure. In yet other alternative embodiments, all or part of base 18 may be removed before a procedure or penetrated before or during a procedure, in order to operate on a patient located under enclosure 12. In some of these embodiments, base 18 may include an adhesive layer that allows a hermetic seal to be formed between base 18 and the patient. These types of embodiments, where the patient is located under the enclosure and the procedure is performed through it, will be described further below. In the embodiment of FIGS. 1A and 1B, enclosure 12 with base 18 provides a completely enclosed environment, accessible only through multiple, seal-forming ports.

A number of features may be included on enclosure 12, such as but not limited to a patient port 14, a first pair of hand ports 16, a second pair of hand ports 22, an upper port 20, and one or more air ventilation ports 24. Patient port 14 is generally a perforated or otherwise seal-forming opening, through which a patient's upper or lower limb is inserted. Patient port 14 has a configuration and is made of a material such that, when the patient's limb is inserted into enclosure 12, patient port 14 automatically forms a seal around a surface of the limb. This seal will help prevent air and contaminants from entering the inside of enclosure 12 during a procedure.

In some embodiments, patient port 14 may also include a built-in, circumferential compression member, which may be used to apply circumferential compression to the surface of the patient's body at or near the same location where the seal is formed. This compression may be used to reduce blood flow to the limb being operated on, thus acting as tourniquet, as is commonly used in surgical procedures on the extremities. The circumferential compression member may be the same component that forms the seal with the patient, or alternatively, system 10 may include separate sealing and compression members. The compression may, for example, be one or more fillable air bladders. Air, liquid or other compression media may be supplied via a built-in or separate compression source, according to various embodiments.

Hand ports 16, 22 may be used by a surgeon or other physician and an assistant (other physician, nurse, etc.) to access the patient's limb. In some embodiments, each of hand ports 16, 22 is configured to form a seal with the user's forearm, in the same way that patient port 14 forms a seal with the patient's limb. Alternatively, as shown in later figures, each hand port 16, 22 may be attached to a built-in surgical glove, so that when the user inserts the hand into one of ports 16, 22, the hand passes immediately into the built-in glove. In the embodiment illustrated in FIGS. 1A and 1B, on the other hand, a surgeon and assistant may gown and glove themselves, as if for a traditional surgical procedure, and then pass their gloved and gowned hands and part of their forearms through hand ports 16, 22.

In some embodiments, some or all of hand ports 16, 22 may be set into enclosure 12 on tracks 17, so that ports 16, 22 can be moved by a user from side to side and/or up and down. Tracks 17 thus allow a user to have more freedom of movement, relative to enclosure 12. Tracks 17 are illustrated schematically in FIG. 1A. In an actual embodiment, tracks may include a slot along which hand ports 16 slide, and a sealing portion for maintaining the sealed environment of enclosure 12 during movement of ports. Tracks 17 are optional, however, and may be excluded from this or other embodiments.

Upper port 20 is another optional feature, which may provide access for an ultraviolet light (or inlet for light) for helping maintain an aseptic and antimicrobial environment, an air purification or clean air flow device, air inflow or air egress from enclosure 12, or any combination thereof. In the embodiment illustrated in FIG. 1A, for example, air inflow into enclosure 12 may be provided via air ventilation port 24, and air outflow may be through upper port 20. Upper port 20 may be configured as a filter, a mesh material, a semipermeable membrane or the like.

Air ventilation port 24 may be include a valve and may be coupled with a device for air input into enclosure 12 or air evacuation from enclosure 12. In many embodiments, as will be described further below, system 10 is configured for generating an air flow through enclosure 12, such as a laminar air flow, positive pressure or other air flow pattern, for helping maintain a sterile or near-sterile surgical environment. In such embodiments, system 10 will include an air inlet port and an air outflow port. In the embodiment of FIGS. 1A and 1B, these ports may be ports 20 and 24. More ideally, in other embodiments described below, air inlet and outlet ports will be two, relatively equally sized ports, to which air hoses or tubes can be connected for passing air into and out of enclosure 12. In alternative embodiments, air ventilation port 24 may be used for other purposes, such as for connecting to suction, irrigation, cautery, or other devices, or as a port for introduction of other instruments into enclosure 12.

FIG. 1B provides a perspective, bottom view of surgical environment enclosure system 10. Base 18, which may also be referred to as a “bottom frame,” may be collapsible and expandable or may have a fixed shape, just as with the rest of enclosure 12. In either case, base 18 may optionally include additional functionality or features, such as but not limited to additional ports or interfaces for equipment, slots and compartments for surgical equipment, built-in instruments, or the like.

Enclosure 12 may be collapsible and expandable in any of a variety of ways and via any of a variety of mechanisms. In one embodiment, for example, enclosure 12 may be made of a shape memory material, such as a shape memory polymer, and it may have a default expanded shape, such as the shape illustrated in FIGS. 1A and 1B. Enclosure 12 may be collapsed and held in a collapsed state within packaging, for example, and when released from the packaging, enclosure 12 may automatically expand back to its default shape. In an alternative embodiment, enclosure 12 may be made of a flexible, inflatable, polymeric material, such that it may be inflated by introduction of air into the inside of enclosure 12. In such an embodiment, and where an air flow is maintained in enclosure 12 for surgical environment purposes, the amount and rate of air passed into the interior of enclosure 12 and the amount and rate of air evacuated from the interior may be balanced in such a way as to maintain enclosure 12 in its inflated/expanded configuration. In yet another embodiment, some or all of the walls of enclosure 12 may be inflatable and deflatable to provide for expansion and collapse. These or any other suitable mechanisms for expansion and/or collapse of enclosure 12 may be used in various embodiments.

System 10 may also include one or more connectors (not shown) for connecting enclosure 12 with a power source, with an air compressor for powering the compression member in patient port 14, with sources of air inflow and outflow, with a HEPA filter or other filter, or the like. To the extent a power source connection is used with system 10, various embodiments may include connections for AC power, DC power or both. Additionally or alternatively, one or more power sources may be included as part of system 10, such as integrated battery pack(s), solar energy collectors or the like. In various embodiments, any feature or component that may be external and connected to enclosure 12 may alternatively be built-in to enclosure 12.

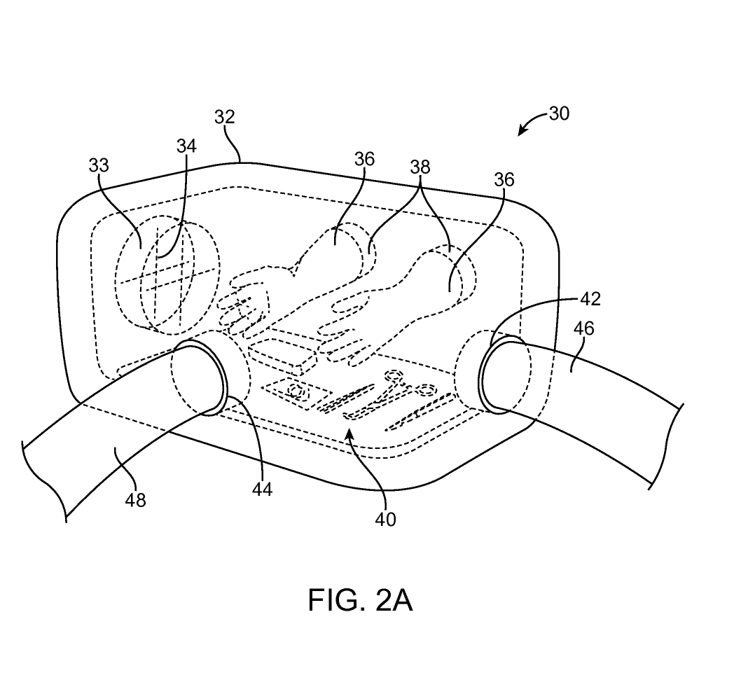

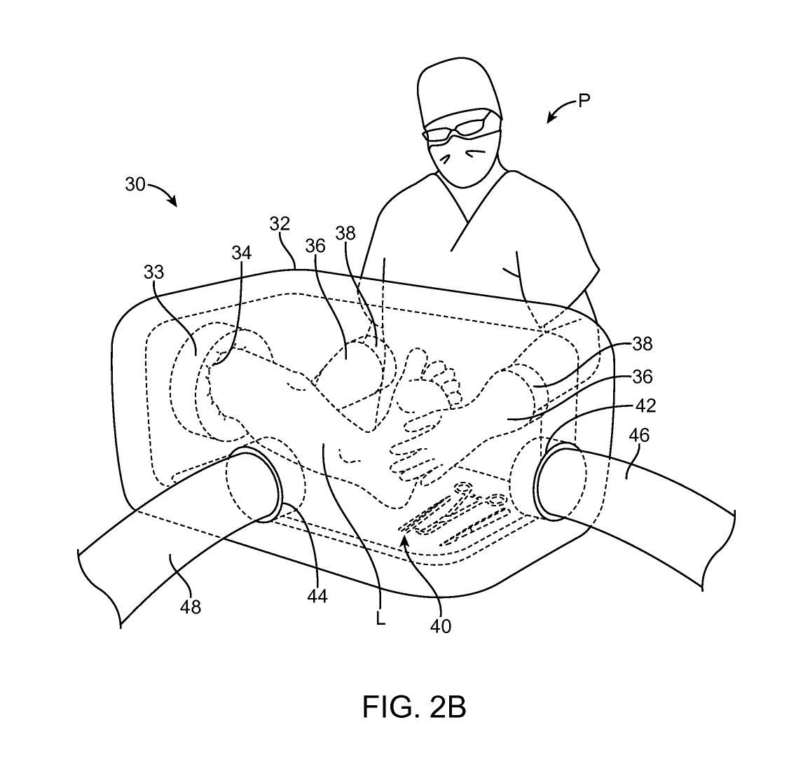

Referring now to FIGS. 2A and 2B, another embodiment of a surgical environment enclosure system 30 is illustrated. FIG. 2B shows system 30 with a physician P and a patient's leg L inserted for performance of a surgical procedure. System 30 may include an enclosure with a patient port 33, having a sealing member 34, two hand ports 38 with built-in gloves 36 for insertion a physician's hands, a first ventilation port 42 connected to a first ventilation tube 46, a second ventilation port 44 connected to a second ventilation tube 48, and surgical instruments 40, which in some embodiments may be prepackaged into enclosure 32. Some of the features and components of this embodiment of system 30 may be similar to those described above, so descriptions will not be repeated.

In this embodiment, system 30 includes two, similarly sized ventilation ports 42, 44, which may be removably coupled with two ventilation tubes 46, 48. In some embodiments, system 30 may be provided with tubes 46, 48, while in alternative embodiments, system 30 may be provided without them, and standard or custom tubes may be provided separately. Ventilation ports 42, 44 and tubes 46, 48 may be used to generate an air flow through enclosure 32, such as a laminar air flow. In some embodiments, tubes 46, 48 may be connected to a filter or filtration system, such as but not limited to a high-efficiency particulate absorption (“HEPA”) filtration system. Either of the combined ventilation ports 42, 44 and tubes 46, 48 may be used for air inflow and air outflow, according to various embodiments.

Built-in gloves 36 are an optional feature and may comprise any suitable glove material, such as but not limited to latex and latex substitute materials. Gloves 36 are attached to ports 38 in a continuous, sealed fashion, to prevent air from entering enclosure through ports 38 during a procedure. Instruments 40 are another optional feature, and any instrument or combination of instruments may be included. As mentioned above, in some embodiments, instruments 40 may be prepackaged into the interior of enclosure 32 before packaging system 30, so that a user opens the package and some or all of the instruments 40 required to perform a procedure are already in enclosure 32. In this way, it is possible to provide customized, procedure-specific surgical environment enclosure systems 30, in which the configuration of enclosure 32, the provided instruments 40, and potentially one or more other features of system 30 are specifically designed for facilitating performance of a given procedure. For example, one embodiment may be designed as a carpal tunnel release surgery system, another may be designed for performing a particular foot surgery, yet another might be for performing a total knee arthroplasty procedure, etc.

As mentioned previously, patient port 33 may include sealing member 34, which may also act as a circumferential compression or tourniquet device to limit blood flow to the leg L (or other limb or body part, in alternative embodiments). In one embodiment, sealing member 34 may serve both as a seal-forming device and as a compression/tourniquet device. In an alternative embodiment, system 30 may include separate sealing and compression/tourniquet members. In some embodiments, sealing member 34 may be inflated using a built-in inflation device attached to or within enclosure 32. Alternatively, sealing member 34 may be coupled with an inflation/compression port for attaching to an air compressor or similar device for providing the compressive force. The compression/tourniquet feature is optional, however, so other embodiments may include a sealing member that serves simply for forming a seal with a surface of the patient's body.

In alternative embodiments, a surgical environment enclosure system, such as those illustrated and described herein, may include two patient ports instead of one. Such embodiments may be designed so that a limb may be inserted into one of the two ports, extend through the inside of the enclosure, and then extend through the other patient port on the other side of the enclosure. In the case of a lower limb, for example, the foot and possibly the ankle might extend out of the second patient port. In this type of embodiment, part of the limb is enclosed in the enclosure during a procedure, while a proximal end of the limb and a distal end of the limb remain outside the enclosure. This configuration may be advantageous in certain clinical scenarios. In some cases, for example, and enclosure may be left intact on a limb for a protracted amount of time, for example to promote healing of a wound or to provide a clean environment if repeated access to a procedure site over time is desired. These embodiments may be beneficial, in such cases, since the patient will still have at least some freedom in his or her distal extremity.

Various processes may be used for providing a sterile, near-sterile or aseptic environment within enclosure 32. Typically, system 30 will be sterilized after packaging and before sale. Additionally or alternatively, system 30 may be provided with a disinfecting or sterilization kit, possibly prepackaged in the interior of enclosure 32, and part of the process of preparing system 30 for use could be to use the sterilization kit to disinfect or sterilize the interior. In some embodiments, as well, system 30 may be capable of sterilization after an initial use and may then be reused for one or more additional procedures after sterilization. In other embodiments, system 30 may be used only for one use and then destroyed. In either case, one method of reusing or destroying system 30 may be to return it to its original packaging or a provided additional package and then mailed to a separate facility for recycling or disposal.

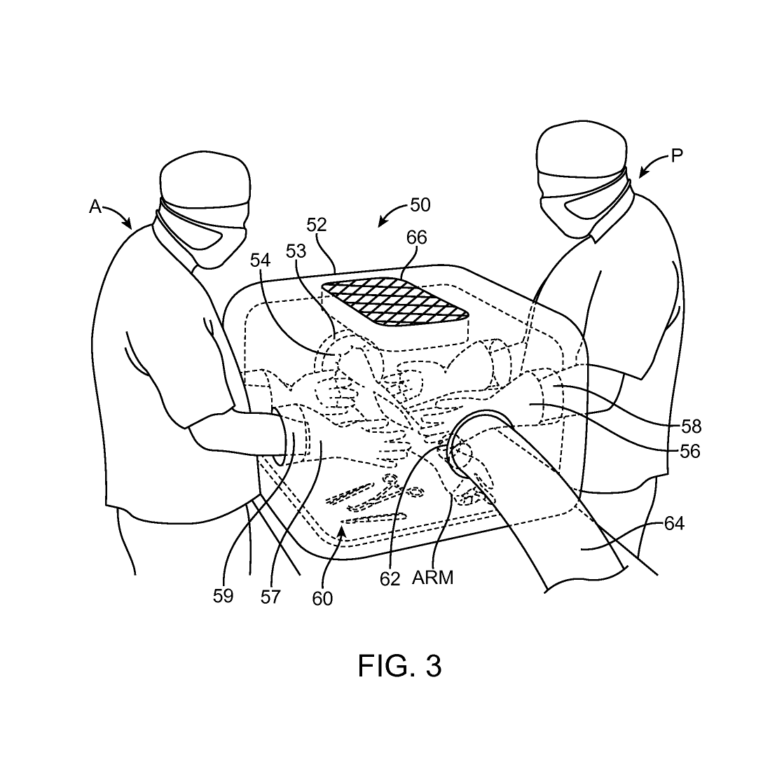

Referring now to FIG. 3, another alternative embodiment of a surgical environment enclosure system 50 may include an enclosure 52, a patient port 53 with a sealing/compression member 54, two pairs of hand ports 58, 59 with two pairs of built-in gloves 56, 57, a side ventilation port 62 attached to a ventilation tube 64, a top ventilation port 66, and prepackaged instruments 60. In FIG. 3, a physician P and an assistant A are operating on an arm of a patient. The physician P in this case is likely a surgeon, and the assistant A may be another surgeon, another physician other than a surgeon, a nurse, a physician's assistant or the like. Of course, any combination of practitioners may use system 50, and in alternative embodiments with greater numbers of hand ports, other physicians and/or assistants may participate in the procedure.

In this embodiment, the air filtration/laminar air flow system may include a central unit (not shown) that is plugged into enclosure via side ventilation port 62 and tube 64. Top ventilation port 66 may be used to allow air to escape enclosure 32. Optionally, top ventilation port 66 may also include a built-in UV light and/or may be used as a port for device insertion.

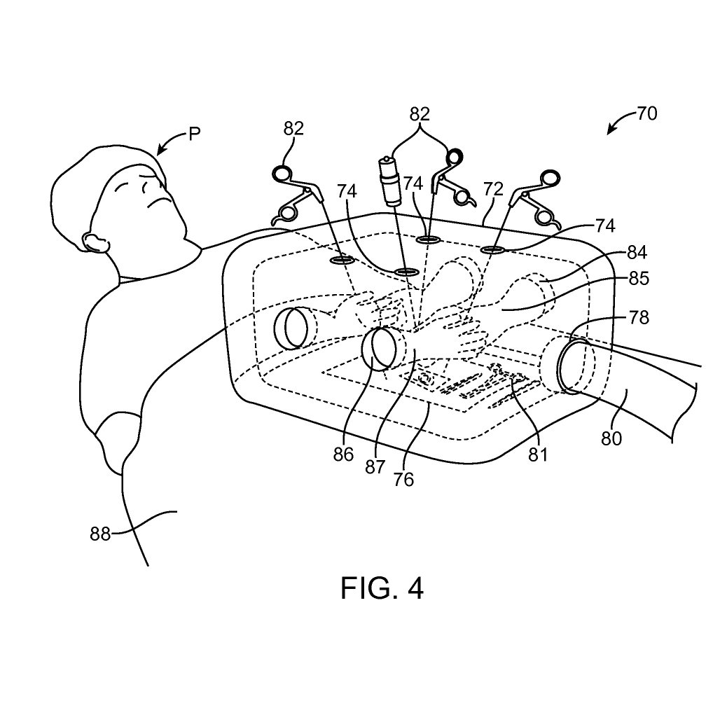

Referring now to FIG. 4, in another embodiment, a surgical environment enclosure system 70 may be configured for use on an abdomen, thorax, back or other location on a patient P—in other words, on a portion of the body that cannot be passed into an enclosure 72 of system 70. In this particular example, system 70 is illustrated being deployed onto a midline laparotomy to facilitate laparoscopic surgery through the bottom of enclosure 72 and within the patient's abdomen. As with previously described embodiments, this use and configuration may be used in lieu of a traditional operating room.

System 70 may include enclosure 72, which houses multiple, self-sealing instrument ports 74, a patient port 76, a ventilation port 78, and two pairs of hand ports 84, 86 coupled with two pairs of built-in gloves 85, 87. System 70 may also be prepackaged with instruments 81, and may also optionally include an air ventilation tube 80 and a custom surgical drape 88, which facilitates draping the patient P in such a way that enclosure 72 can be easily applied. This embodiment may also include an additional air ventilation port (not shown) to allow for generation of a laminar air flow. Any number, location and size of instrument ports 74 may be included. In this embodiment, enclosure 72 is open on the bottom, patient-contacting surface, and this opening acts as patient port 76. In some embodiments, all or a portion of the patient-contacting surface of enclosure 72 may include an adhesive to attach to surgical drape 88 or directly to the patient's skin. The patient's skin may be cleaned and disinfected before and/or after placement of enclosure 72 on the patient P. Instruments 81 may be prepackaged within enclosure 72, or alternatively, an entire procedure may be performed with laparoscopic instruments 82 through instrument ports 74.

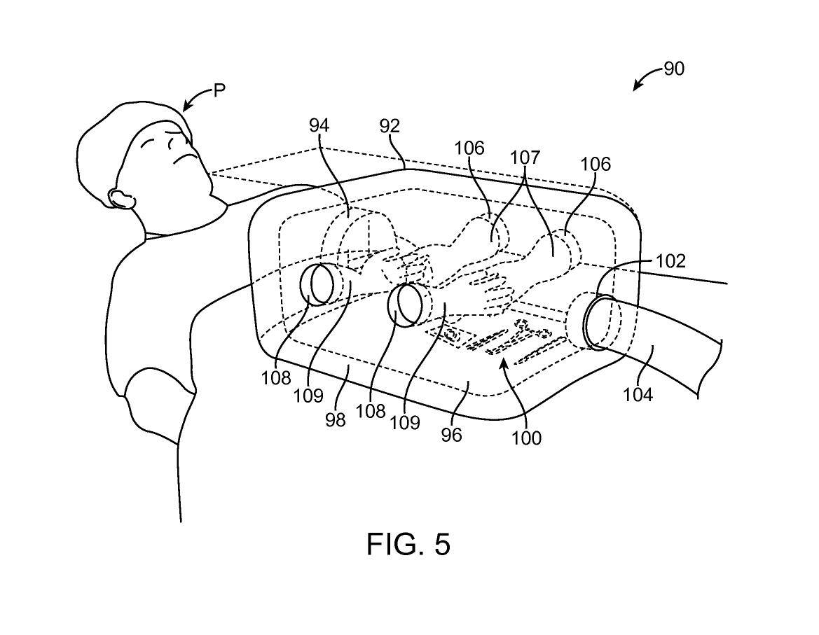

With reference now to FIG. 5, in another embodiment, a surgical environment enclosure system 90 may include an enclosure 92, a first patient port 94, a second patient port 96, two pairs of hand ports 106, 108, two pairs of built-in gloves 107, 109, an adhesive strip 98, prepackaged instruments 100, an air ventilation port 102 and an air ventilation tube 104. Additionally, system 90 will generally include a second air ventilation port (not shown), which can be used with air ventilation port 102 to create a laminar air flow. Adhesive strip 98 may be located on the base of enclosure 92 or on a surgical drape custom designed to be used with enclosure 92. This embodiment includes two patient ports 94, 96, so that it may be used for any of a number of different surgical or medical procedures. If first patient port 94 is used, in a limb-based procedure for example, the bottom of enclosure may remained closed and sealed at all times. If second patient port 96 is used, such as for an open abdominal procedure, second port 96 is open and first port 94 typically remains closed. In the embodiment shown in FIG. 5, second port 96 is somewhat larger than port 76 in FIG. 4. This may be the configuration used, for example, in open procedures rather than laparoscopic procedures. This difference illustrates that various embodiments of surgical environment enclosure systems may be used for any of a number of different surgical or medical procedures on any of a number of body parts.

In one embodiment, a method for using a surgical environment enclosure for performing a medical or surgical procedure may first involve one or more preparatory steps. For example, initially, the patient may be prepared in any way necessary for the procedure, such as application of general and/or local anesthetic, cleaning, prepping and draping the surgical site, and the like. At the same time, before or after this step, the surgical environment enclosure system may be prepared for use. For example, the system may be removed from packaging and allowed to expand or made to expand. Expansion may be achieved via the system's own energy source or by plugging it into an electrical outlet or other energy source, air source, or other means of expansion. The system may also be prepared in any of a number of additional ways, such as attaching air inflow and outflow tubes to air inflow and outflow ports and generating a laminar air flow through the enclosure, activating self-contained air filtration devices, depositing one or more sterile surgical instruments into the enclosure, and/or attaching to one or more additional devices, such as cautery, irrigation, suction, and/or any other devices.

In the example of a carpal tunnel release surgery, the forearm and hand may be prepared in the usual manner, with local anesthesia being administered to the median nerve for a nerve block prior to prepping and draping, or after, or after insertion into the surgical environment, as desired. The surgeon and/or assistant(s) may access the surgical environment enclosure, either through built-in gloves or by inserting scrubbed their gloved/gowned hands into the hand ports. The patient's arm may be advanced into the enclosure at any suitable time, through the patient port, and the sealing/compression member may be used to compress the upper arm and thus limit blood flow to the hand. The surgeon may then perform the procedure, optionally with the help of the assistant. When the procedure is completed, the patient's arm may be removed from the enclosure, or in some embodiments, the enclosure may be dissembled. Depending on the embodiment, the system may then either be disposed of or processed by recycling and re-sterilization for future procedure(s).

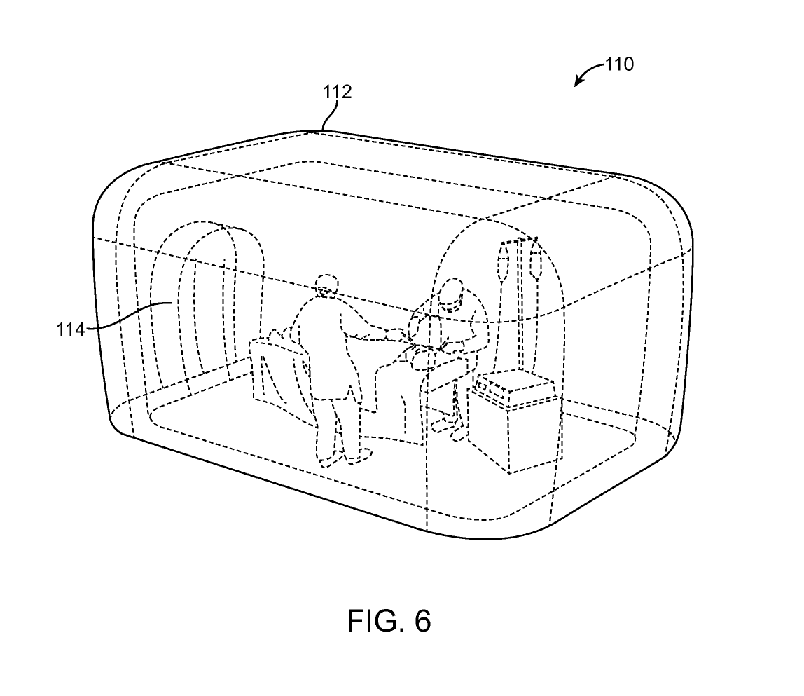

With reference now to FIG. 6, in a significantly different embodiment than those described above, a surgical environment enclosure system 110 may be large enough to act as a complete operating theater/operating room. Such a system 110 may include an enclosure 112, a door 114 and some of the features described above, such as an air circulation system. In this embodiment, the patient, the surgeon, one or more assistants, other equipment, and the like fit within enclosure 112. System 110 may be used to maintain a clean, operating-room-quality environment at much lower cost than building a standard operation room. Door 114 and other features of system 110 may help provide a relatively germ free environment. Such an embodiment may be very useful in mobile crisis situations, such as battlefields or natural disasters, as a significantly less expensive surgical environment than a traditional operating room, as a substitute for an operating room during construction or renovation of a hospital or surgery center, as a solution in remote, third-world geographies, or the like. In various embodiments, it may be easily disinfected or sterilized using common techniques. It may be used for a specified number of procedures or may be used indefinitely, according to various embodiments.

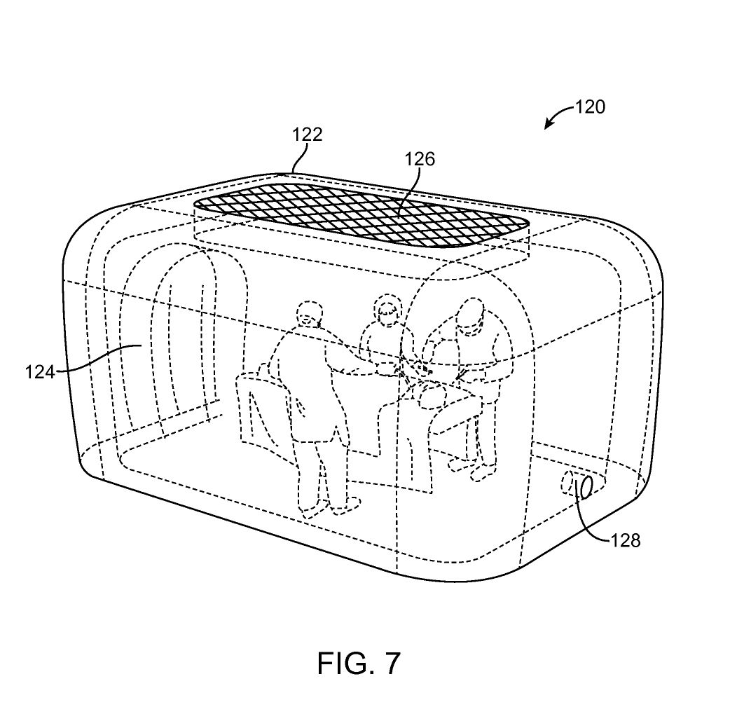

Referring to FIG. 7, an embodiment of a surgical environment enclosure system 120 is similar to that illustrated in FIG. 6. System 120 may include an enclosure 122, a door 124, an air inlet port 128, an air outlet ventilation filter 126, and any of a number of additional features.

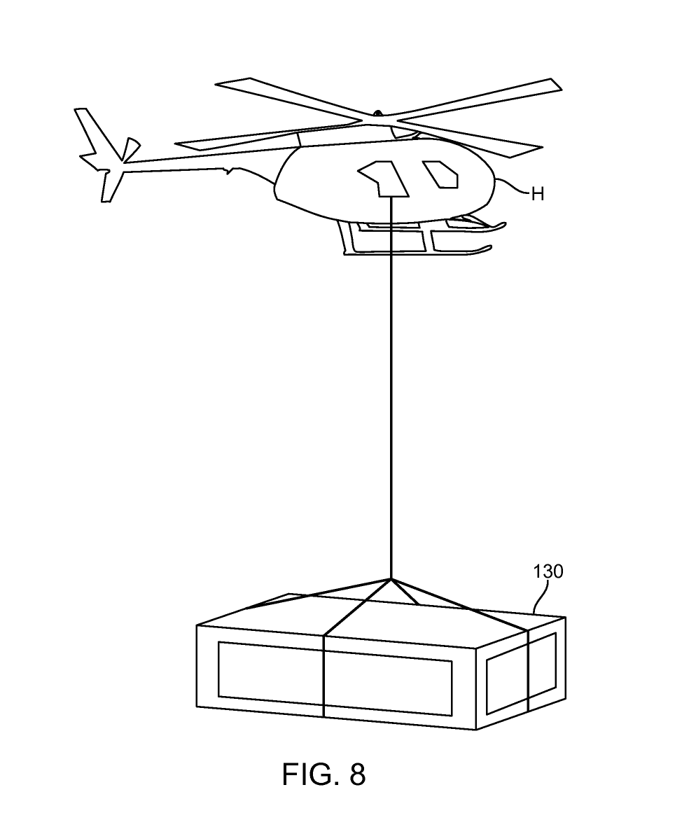

FIG. 8 illustrates the fact that in some embodiments, a surgical environment enclosure system, such as those described in relation to FIGS. 6 and 7, may be collapsed and enclosed in packaging 130, which may then be transported by any of a number of suitable means. Packaging 130, in this example, is being transported by helicopter H, as may be needed during an airlift of supplies into a disaster area or battlefield location, for example. Of course, any other transportation may be used as well.

Although this detailed description has explained a number of embodiments and features in detail, this description is meant to be exemplary and not exclusive in nature. Various embodiments may include alterations without departing from the scope of the invention as it is set forth in the claims. Thus, the scope of the present invention should not be limited by the particular disclosed embodiments described above, but should be determined only by a fair reading of the claims that follow.

What is claimed is:

1. A method for performing a surgical or medical procedure on a portion of a patient's body within a surgical environment enclosure, the method comprising:
preparing the surgical environment enclosure for performing the procedure;
advancing the portion of the patient's body into the surgical environment enclosure through a first port on the enclosure, wherein the first port forms a seal around a surface of the patient's body; and
performing the surgical or medical procedure on the portion of the patient's body inside the surgical environment enclosure, through at least a second port on the enclosure,
wherein neither the entire body of the patient nor an entire body of any medical or surgical personnel fully enters the surgical environment enclosure during performance of the surgical or medical procedure.

2. The method of claim 1, wherein preparing the surgical environment enclosure comprises expanding the enclosure from a collapsed configuration to an expanded configuration.

3. The method of claim 2, wherein preparing the surgical environment enclosure comprises removing the enclosure from sterile packaging before expanding the enclosure.

4. The method of claim 3, wherein expanding the enclosure comprises releasing the enclosure from the sterile packaging, wherein the enclosure automatically expands to a default configuration when released from constraint.

5. The method of claim 2, wherein expanding the enclosure comprises inflating a portion of the enclosure.

6. The method of claim 1, wherein preparing the surgical environment enclosure comprises establishing an air flow through the enclosure by passing air into the enclosure through a first ventilation port and passing air out of the enclosure through a second ventilation port.

7. The method of claim 1, wherein preparing the surgical environment enclosure comprises passing at least one sterile instrument into the enclosure.

8. The method of claim 1, wherein preparing the surgical environment enclosure does not require passing any instruments into the enclosure, because the enclosure is provided with at least one instrument prepackaged within it.

9. The method of claim 1, wherein advancing the portion of the patient's body into the enclosure comprises passing an extremity of the patient's body at least partway into the enclosure.

10. The method of claim 9, wherein advancing the extremity of the patient's body comprises:
advancing the extremity through the first port; and
advancing a distal end of the extremity through a second port, so that a proximal end and a distal end of the extremity reside outside of the enclosure during the procedure.

11. The method of claim 9, wherein the extremity comprises an arm.

12. The method of claim 11, wherein performing the procedure comprises performing a carpal tunnel release procedure.

13. The method of claim 9, further comprising forming a blood flow limiting compression around the surface of the patient's body using a compression member coupled with the first port.

14. The method of claim 1, wherein performing the procedure comprises extending two hands of a surgeon into the enclosure through the second port and a third port on the enclosure.

15. The method of claim 14, wherein performing the procedure further comprises extending two hands of an assistant into the enclosure through a fourth port and a fifth port on the enclosure.

16. The method of claim 14, wherein passing the hands of the surgeon into the enclosure comprises passing the hands into gloves that are attached to the second and third ports.

17. The method of claim 1, wherein performing the procedure comprises passing a working distal end of at least one instrument through an instrument port on the enclosure, wherein a proximal end of the at least one instrument remains outside the enclosure for grasping and manipulating by a surgeon or assistant.

18. The method of claim 1, further comprising, after performing the procedure:
collapsing the enclosure; and
disposing of the enclosure.

19. The method of claim 18, wherein collapsing the enclosure comprises removing air from a portion of the enclosure.

20. The method of claim 18, wherein disposing of the enclosure comprises mailing the enclosure to a facility for disposal.

21. The method of claim 1, further comprising, after performing the procedure:
sterilizing the enclosure; and
reusing the enclosure for an additional procedure.

22. The method of claim 1, wherein preparing the surgical environment enclosure comprises sterilizing an internal surface of the enclosure.

23. A surgical environment enclosure system for performing a surgical or medical procedure on a portion of a patient's body, the system comprising:
an enclosure;
a patient port on the enclosure for allowing passage of the portion of the patient's body into the enclosure, wherein the patient port includes a seal-forming member for forming a seal with a surface of the patient's body to prevent air from passing into the enclosure through the patient port while the portion of the patient's body is in the enclosure;
at least two hand ports on the enclosure for allowing passage of two physician hands into the enclosure to perform the procedure; and
at least a first ventilation port on the enclosure for allowing controlled passage of air into the enclosure.

24. The system of claim 23, wherein the enclosure is collapsible and expandable.

25. The system of claim 23, wherein the enclosure, when in an expanded state, is at least partially rigid.

26. The system of claim 23, wherein the enclosure comprises a transparent material.

27. The system of claim 23, wherein an internal surface of the enclosure is sterile.

28. The system of claim 23, wherein the at least two hand ports comprise four hand ports for allowing passage of two physician hands and two assistant hands into the enclosure.

29. The system of claim 23, wherein the at least two hand ports are moveable along a surface of the enclosure to allow the physician's hands to move in a direction parallel to the surface of the enclosure.

30. The system of claim 29, further comprising a track in the surface of the enclosure, wherein the at least two hand ports move along the track in at least two directions along the surface.

31. The system of claim 23, further comprising at least two built-in gloves, wherein each of the at least two hand ports is attached to one of the built-in gloves extending into the enclosure such that when one of the hands passes through one of the hand ports it passes immediately into the glove attached to that port.

32. The system of claim 23, further comprising a circumferential compression member coupled with the patient port to apply circumferential compression to a surface of the patient's body and thus limit blood flow to the portion of the patient's body located within the enclosure.

33. The system of claim 23, further comprising an air input tube removably coupled with the first ventilation port.

34. The system of claim 23, further comprising a second ventilation port on the enclosure for allowing controlled passage of air out of the enclosure.

35. The system of claim 34, further comprising:
an air input tube removably coupled with the first ventilation port; and
an air evacuation tube removably coupled with the second ventilation port.

36. The system of claim 35, further comprising an air circulation device coupled with the air input tube and the air evacuation tube for providing air to, and removing air from, the enclosure.

37. The system of claim 36, wherein the air circulation device comprises a high-efficiency particulate absorption filtration system.

38. The system of claim 23, further comprising at least one instrument port on the enclosure for allowing at least one surgical instrument to be passed therethrough, wherein the at least one instrument port is configured to form a seal with the at least one surgical instrument.

39. The system of claim 23, further comprising an additional patient port on the enclosure, configured to allow passage of a distal end of one of the patient's limbs therethrough, so that a proximal end and the distal end of the patient's limb reside outside the enclosure during the procedure.

40. The system of claim 23, further comprising at least one instrument prepackaged within the enclosure for use in the medical or surgical procedure.
